(19) United States
(12) Patent Application Publication
(10) Pub. No.: US 2005/0186278 A1
(43) Pub. Date: Aug. 25, 2005

(54) Gastroprotected formulations containing alph-amylase inhibitors

(76) Inventor: Di Pierro Francesco, Trezzano Sul Naviglio (IT)

Correspondence Address: Walter H. Schneider, 21530 Beechwood Rd., Circleville, OH, 43113, US

(21) Appl. No.: 11/046,952

(22) Filed: Jan. 31, 2005

(30) Foreign Application Priority Data
Feb. 24, 2004 (IT) MI2004A000313

(51) Int. Cl. A61K 35/80; A61K 9/24; A61K 35/78; A61K 31/555; A61K 38/16

(52) U.S. Cl. 424/472; 424/195.17; 424/757; 514/12; 514/55; 514/184

(57) ABSTRACT

Disclosed are gastroresistant formulations of phaseolamin characterized in that the enteric coating consists of shellac or other coatings compatible with nutritional use.

The present invention relates to gastroprotected formulations containing protein substances that inhibit alpha-amylase, optionally in combination with other ingredients with antiglycemic, antidiabetic (type II) and antiobesity activity.

As is well known, diet in socially, economically developed countries markedly increases the percentage of incidence of disorders related to overweight, obesity and type II diabetes.

The common feature of these disorders is an excessive increase of the glycemic curve, which accounts for the majority of the consequences.

Drastic changes in alimentary habits would indeed determine marked improvements; however, medicaments able to support any desirable (but not always possible) dietetic intervention are still necessary.

Ingestion of complex carbohydrates (pasta, bread, rice, potatoes, etc.) is known to determine the production of pancreatic alpha-amylase, an enzyme that digests starches, i.e. glucose polymers having from 3 to 9 sub-units. Intestinal maltases and alpha-dextrinases subsequently digest short polymers to free glucose units that, in the blood, give rise to the so called glycemic curve. As a consequence of glucose increase, insulin raises up to a peak, acting as a scavenger that cleans up the blood from free glucose either directing it to the brain or storing it in the muscle and in the liver as glycogen rosettes. Since the muscle and the liver are often already full of rosettes, insulin transforms free glucose in fat deposits (fat tissue).

Alpha-amylase inhibitors able to block upstream the above-described cascade are known. Phaseolamin, a thermolabile and gastrolabile protein of about 55 KD, obtained by extraction with hot water from the fruit of Phaseolus vulgaris (common bean) is known to strongly inhibit alpha-amylase already at very low molarities.

Phaseolamin with various degrees of specific activity is commercially available, depending on the commercial source, and is contained in different supplements or dietary products.

It has now been found that phaseolamin suitably formulated as a gastroprotected form is more active and allows to reduce the daily dosage or the use of a phaseolamin with not particularly high specific activity.

Gastroprotection is obtained by means of a shellac coating (or a coating of any other compatible polymer having the same function as shellac and also complying with the regulations concerning nutritional use) which allows the enzyme to be released in the intestine in the active form (98%), thus inhibiting pancreatic alpha-amylase (released in the intestine) and blocking the cascade of events that usually increases the glycemic curve.

Shellac usually ranges from about 1 to about 10% of the formulation weight.

Gastroprotected forms are prepared according to conventional techniques, which comprise spraying tablets or microgranules (prepared with known methods) with a shellac aqueous suspension or solution together with antiadhesives (talc, lactose, microcrystalline silica, magnesium stearate), plasticizers (PVP, glycerol, fatty acids) and optionally dyes or pigments.

The formulations of the invention, which will contain from about 1 to about 100 mg, preferably from about 5 to about 20 mg, of phaseolamin per dose unit, will be in the form of coated tablets, microgranules, multilayer tablets, granulates and the like.

Further to phaseolamin, the formulations of the invention will contain, if desired, other active ingredients, such as the aqueous, ethanol or aqueous-ethanol extracts of Eugenia Jambolana seeds (also known as Jambul, endowed with high anti-type II diabetes activity), Fucus seaweeds, chitosan, mineral salts and oligoelements (in particular chromium in its various stable forms).

The in vitro pharmacological profile, the toxicological study on animals and the clinical evaluation aimed at determining the compliance and the tolerability of the formulations of the invention gave better, surprising results compared with non-gastroprotected conventional forms, in particular in the control of glycemia and overweight.

The following examples illustrate the invention in greater detail.

### EXAMPLE 1

Gastroprotected tablets

| COMPONENT | mg/cpr | % |
|---|---|---|
| Calcium phosphate | 50.551 | 54.947 |
| Microcrystalline cellulose | 25.000 | 27.174 |
| Phaseolamin | 10.000 | 10.870 |
| Shellac | 3.469 | 3.771 |
| Magnesium stearate | 0.900 | 0.978 |
| Glycerine | 0.314 | 0.341 |
| Silicon dioxide | 0.900 | 0.978 |
| Talc | 0.208 | 0.226 |
| Lactose | 0.208 | 0.226 |
| Acetyl monoglycerides | 0.219 | 0.238 |
| Hydroxypropyl methyl cellulose | 0.104 | 0.113 |
| Polyvinylpyrrolidone | 0.073 | 0.079 |
| Dye E124 | 0.055 | 0.059 |
| TOTAL | 92.000 | |

### EXAMPLE 2

Gastroprotected tablets

| COMPONENT | mg/cpr | % |
|---|---|---|
| Calcium phosphate | 68.932 | 19.695 |
| Microcrystalline cellulose | 150.000 | 42.857 |
| Phaseolamin | 10.000 | 2.857 |
| E. Jambolana | 100.000 | 28.571 |
| Shellac | 13.200 | 3.771 |
| Magnesium stearate | 0.850 | 0.243 |
| Glycerine | 1.192 | 0.341 |
| Silicon dioxide | 3.500 | 1.000 |
| Talc | 0.792 | 0.226 |
| Lactose | 0.792 | 0.226 |
| Acetyl monoglycerides | 0.219 | 0.062 |
| Hydroxypropyl methyl cellulose | 0.396 | 0.113 |
| Polyvinylpyrrolidone | 0.073 | 0.021 |
| Dye E124 | 0.055 | 0.016 |
| TOTAL | 350.000 | |

1. Gastroprotected formulations containing phaseolamin characterized in that the enteric coating consists of shellac or other coating compatible with nutritional use.

2. The formulations as claimed in claim 1 in the form of coated tablets or microgranules.

3. The formulations as claimed in claim 2 containing from 1 to 100 mg. of phaseolamin per dose unit.

4. The formulations as claimed in any of claim 3 further containing one or more of the following components: E. Jambolana, Fucus Seaweeds, chitosan, salts, vitamins, chromium picolinate.

5. (canceled)

6. A process for controlling glycemia and over-weight which comprises medicating a patient with an effective dosage of a formulation according to claim 4.
